(19) United States
(12) Patent Application Publication
(10) Pub. No.: US 2018/0185270 A1
(43) Pub. Date: Jul. 5, 2018

(54) MUCOSAL DELIVERY OF VITAMIN B12

(71) Applicant: ZIM LABORATORIES LIMITED, Kalmeshwar, Maharashtra (IN)

(72) Inventors: Anwar DAUD, Kalmeshwar (IN); Nidhi SAPKAL, Kalmeshwar (IN); Lubna DAUD, Kalmeshwar (IN); Minal BONDE, Kalmeshwar (IN)

(73) Assignee: ZIM LABORATORIES LIMITED, Kalmeshwar, Maharashtra (IN)

(21) Appl. No.: 15/580,684

(22) PCT Filed: Jun. 4, 2016
(86) PCT No.: PCT/IN2016/050168

§ 371 Date: Dec. 7, 2017

(30) Foreign Application Priority Data
Jun. 8, 2015 (IN) 2195/MUM/2015

(51) Int. Cl. A61K 9/00; A61K 31/714; A61K 9/20

(52) U.S. Cl. CPC A61K 9/006; A61K 31/714; A61K 9/2095; A61K 9/2013; A61K 9/205

(57) ABSTRACT

This invention comprises a method of transmucosal delivery of process of Vitamin B12 without the need of intrinsic factor comprising administering a solid composition comprising a Vitamin B12 and at least one bifunctional macromolecule with hydrophilic exterior and with hydrophobic pockets capable of pocketing Vitamin B12 material, illustrated by cyclodextrin, at least one permeation enhancer, illustrated by Isopropyl Myristate and at least one agent that is mucoadhessive as well as penetration enhancer, illustrated by chitosan. The solid composition of Vitamin B12 of claim 2 may comprise a lozenge, a candy, a wafer, a tablet, a patch, a film, a spray, a lip balm, or gum.

### TECHNICAL FIELD

The invention relates to dosage forms for delivery of Vitamin B12 and its derivatives.

### BACKGROUND OF THE INVENTION

Oral Methylcobalamin absorption is done by a very specific receptor mediated transport system from the distal ileum after complexation with intrinsic factor. In case of intrinsic factor deficiency in pernicious anemia or due to ageing in 10-30% of the ageing people, patients suffer from the effects of Vitamin B12 deficiency. In such patients, oral Vitamin B12 supplementation is of no use on account of inability to absorb the orally consumed Vitamin B12 and the patient has to rely on painful injections. Vitamin B12 deficiency may also arise in strict vegetarians on account of low intake of Vitamin B12. In these cases too Vitamin B12 in serum or plasma at a low value of 120 to 180 pmol/L (170 to 250 pg/mL) may represent a long term abnormality (Beck 1991) because as deficiency develops, serum values may be maintained at the expense of B12 in the tissues. Thus, a serum B12 value above the cutoff point does not necessarily indicate adequate B12 status and, a far larger intake than the Reference Daily Intake (RDI) would be required to first replenish the tissue deficiency and then to elevate the serum content of Vitamin B12. Hence, even in case of human subjects who have not lost the intrinsic factor and are capable of absorbing Vitamin B12 provided orally, the requirement of fulfilling the deficiency may still far exceed RDI; and on account of limitations on the quantity of intrinsic factor even in normal individuals, ability to absorb orally administered Vitamin B12 very rapidly declines and oral dosing exceeding several times the RDI does not help in improving Vitamin B12 levels fast enough to reach the normal levels in cases wherein the deficiency may be of a high magnitude. It is widely regarded that a B12 content of 1.5 to 2.5≈ [symbol] /meal saturates ileal receptors and thus limits further absorption. Total absorption increases only to a limited extent with increasing intake. A dams et al (1971) reported that nearly 50 percent was retained at a 1 ιg dose, 20 percent at a 5-ιg dose, and just over 5 percent at a 25-ιg dose. It is also reported by Heyssel et al., 1966 that the second of two doses of B12 given 4 to 6 hours apart is absorbed as well as the first indicating utility of giving split doses of Vitamin B12 in patients with normal levels of intrinsic factor. When large doses of crystalline B12 are ingested, up to approximately 1 percent of the dose may be absorbed by mass action even in the absence of intrinsic factor (Berlin et al. 1968; Doscherholmen and Hagen, 1957). This makes it essential to rely only on injections of Vitamin B12, which are painful; and may be useful as a temporary measure to alleviate high deficiency. However, after alleviating the deficiency, injections can not be relied upon for daily maintenance dosing, which may be required for patients having impaired function relating to intrinsic factor. Hence, there exists a need for a method and dosage form for oral dosing of Vitamin B12 which is not dependent on intrinsic factor for absorption; which would at least be useful for providing maintenance dosing to intrinsic factor deficient individuals after replenishment of the normal levels of Vitamin B12 injections; and at the best, a dosage form that has so much improvement in absorption independent of intrinsic factor that it may be possible to avoid Vitamin B12 injections even for replenishment of the deficiency, if the improved dosage form is capable of rapidly increasing the absorption of orally administered Vitamin B12.

EP 2632430 A1 (text from WO2012056299A1) disclosed intranasal formulations of vitamin B derivatives such as methylcobalamin, The stable intranasal aqueous compositions comprise methylcobalamin or cynocobalamin in concentration from 500 mcg/0.1 ml to 1500 mcg/0.1 ml, co-solvents/solubilizers or mixtures thereof in water, and optionally with penetration enhancers, and optionally preservatives, mucoadhesive agents, chelating agents, humectants, antioxidants, or combination thereof, and wherein the pH of the composition is 5 to 7 and viscosity of 1 to 200 Cps. Bile salts such as sodium glycocholate were used as penetration enhancers. Marked increase was seen in trans nasal penetration of formulation containing sodium glycholate as compared to formulation in which sodium glycocholate is absent. It was also concluded that the optimum concentration of sodium glycocholate showing highest penetration was 1% (c.f. 2% or 1.5%). WO2007103931 discloses a method for administration of a nanofluidized nanosuspension containing vitamin B-12 to a subject via a transmucosal route comprising: forming, via a nanofluidization process, a stable nanosuspension comprising nanodroplets of said vitamin B-12; and contacting said nanosuspension with the oral mucosal membranes of said subject; wherein said nanosuspension containing vitamin B-12 is absorbed into the bloodstream of said subject. WO2007103931 also discloses a method for administration of a nanofluidized suspension containing vitamin B-12 to a subject comprising: forming, via a nanofluidization process, a stable nanosuspension comprising nanodroplets of said vitamin B-12; and administering said nanosuspension to said subject; wherein said nanosuspension containing vitamin B-12 is absorbed into the bloodstream of said subject.

WO2007103931 further discloses a method for ameliorating symptoms of pernicious anemia in a subject displaying said symptoms comprising: providing a nanosuspension containing vitamin B-12 in a size range of about 87 nm to about 10 [symbol] ; and contacting said nanosuspension with the oral mucosal membranes of said subject; wherein said nanosuspension containing vitamin B-12 is absorbed into the bloodstream of said subject.

WO2007103931 still further discloses an in vivo process for accelerated formation, maturation, and normalization of red blood cells comprising: providing a nanosuspension containing vitamin B-12 in a size range of about 87 nm to about 10 [symbol] ; and contacting said nanosuspension with the oral mucosal membranes of a subject, whereby said nanosuspension containing vitamin B-12 is absorbed into the bloodstream of said subject wherein mature red blood cells of normal size and shaped are formulated within an accelerated ti me period.

U.S. Pat. No. 3,060,095 has disclosed a method of administering a vitamin B12 material which comprises orally administering said vitamin B12 material as an adsorbate on magnesium trisilicate as the carrier. U.S. Pat. No. 3,060,095 has also claimed that vitamin B12 material can be cyanocobalamine or hydroxycobalamine also.

US2013149255 has disclosed a composition, comprising: a. cyanocobalamin, hydroxocobalamin, methylcobalamin in substantially equivalent ratios; and b. a carrier suitable for forming a solid or semi-solid carrier matrix. The composition is formulated as a lozenge, a candy, a wafer, a tablet, a patch, a film, a spray, a lip balm, or gum.

US20080160070 has disclosed a transdermal vitamin B12 delivery patch that is applied to the skin of a user for the delivery of vitamin B12 to the bloodstream of the user, said patch comprising: a fabric backing; and a skin-adhesive polymer matrix attached to one side of said fabric backing, said matrix containing a vitamin B12 compound, whereby said compound diffuses from the matrix through the stratum corneum layer of the user's skin, through the dermis layer of the ski n, and into the user's bloodstream.

WO2012122313 has disclosed a nanoparticle or a micelle, or a liposome containing micelle comprising a therapeutic agent encapsulated by one or more polymer(s) to which vitamin B12 or a derivative thereof is attached to the at least one or more polymer(s) via a linker group. WO2011130716 has disclosed a nanoparticle comprising: one or more synthetic or natural polymers comprising one or more charged and/or ionisable groups, a therapeutic agent comprising one or more charged and/or ionisable groups of the opposite charge to the charge of the polymers, and, a vitamin B12 covalently linked to the nanoparticle via an optional linker group.

U.S. Pat. No. 4,432,975 has disclosed a process for enhancing the absorption of Vitamin B-12 into the bloodstream, comprising administering Vitamin B-12 sublingually as a micro-lozenge containing from about 0.1% to about 10% by weight cyanocobalamin or hydroxocobalamin. U.S. Pat. No. 4,432,975 has also disclosed process for enhancing the absorption of Vitamin B-12 into the bloodstream, comprising administering Vitamin B-12 sublingually as a micro-lozenge comprising from about 0.1% to about 10% by weight crystalline cyanocobalamin or hydroxycobalamin, about 0.1% to about 5% of a lubricant selected from the group consisting of magnesium stearate and hydrogenated vegetable oils, and a pharmacologically acceptable carrier. Also disclosed is a micro-lozenge composition for introducing Vitamin B-12 sublingually into the bloodstream comprising about 0.1 to about 10 percent by weight of crystalline cyanocobalamin or hydroxycobalamin, about 0.1 to about 5 percent by weight of a lubricant selected from the group consisting of magnesium stearate and hydrogenated vegetable oils, approximately 0.1 to about 5 weight percent alginic acid, approximately 0.1 to about 5 weight percent polyethylene glycol and a pharmacologically acceptable carrier.

J on D. Zeltman EP 2124907 A2 (text from WO2008116004A2, 25 Sep. 2008) has disclosed a shelf stable transdermal delivery patch for administering vitamin B₁₂ to a subject, comprising (a) a backing layer, (b) a skin contact adhesive layer adjacent to the backing layer, said skin contact adhesive layer including (I) a polymeric adhesive, (II) vitamin B₁₂, (III) a penetration enhancer effective to enhance transdermal uptake of vitamin B₁₂ by said subject, and (iv) a vitamin B₁₂ stabilizer to stabilize the vitamin B₁₂ contained within the adhesive layer, and (c) a removable impermeable layer overlaying the skin contact adhesive layer, said removable impermeable layer preventing vitamin B₁₂ release from the skin contact adhesive layer prior to use

### SUMMARY OF THE INVENTION

This invention comprises a solid composition of Vitamin B12 for transmucosal delivery without the need of intrinsic factor; the composition comprising Vitamin B12 and, at least one bifunctional macromolecule with hydrophilic exterior and with hydrophobic pockets capable of pocketing Vitamin B12 material, at least one permeation/penetration enhancer and at least one agent that is mucoadhessive as well as permeation/penetration enhancer. The Vitamin B12 material comprises, at least one or more, selected from the group consisting of cyanocobalamin, hydroxocobalamin and methylcobalamin, the macromolecule with hydrophilic exterior and with hydrophobic pockets capable of pocketing Vitamin B12 material comprises, at least one or more, selected from the group comprising cyclodextrin and its derivatives, the permeation/penetration enhancer comprises, at least one or more, selected from the group consisting of Isopropyl Myristate, glycerol myristate, myristic acid and their derivatives or any other fatty acid esters that has permeation enhancement ability, the mucoadhesive as well as permeation/penetration enhancer comprises, at least one or more, selected from the group consisting of chitosan, trimethyl chitosan (TMC), dimethylethyl chitosan (DMEC), diethylmethyl chitosan (DEMC), triethyl chitosan (TEC) and any derivative of chitosan or any substituted polysaccharides that has both mucoadhesive and permeation enhancement ability.

The solid composition of Vitamin B12 of claim 2 may comprise a lozenge, a candy, a wafer, a tablet, a patch, a film, a spray, a lip balm, or gum. The solid composition of Vitamin B12 may be: (a) a film, more particularly, a sub-lingual film further comprising a film forming polymer, propylene glycol or any other plasticizer, sucralose or any other high intensity sweetener, and Magnesium aluminium silicate or any other antisticking, anti-tacky agent, (b) a tablet or (c) a lozenge further comprising a bulking agent, a disintegrant, a lubrincant, a high intensity sweetener, binder, antiadherent and other excipient/s and the like. The other excipients may include diluents, glidants, superdisintegrants, flavoring agents, taste modifiers, taste masking agents, mucoadhesive agents, buffering agents, stabilizers, preservatives and the like.

The solid composition of Vitamin B12 comprises [symbol] -Cyclodextrin 2.5 to 15% of the composition, Isopropyl Myristate 0.5 to 15% of the composition and chitosan 1 to 15% of the composition.

This invention also embodies a method of transmucosal delivery of process of Vitamin B12 without the need of intrinsic factor comprising administering a solid composition comprising Vitamin B12 material comprising, at least, one bifunctional macromolecule with hydrophilic exterior and with hydrophobic pocket capable of pocketing Vitamin B12 material, at least one permeation/penetration enhancer and at least one agent that is mucoadhessive as well as permeation/penetration enhancer.

This invention also embodies a process of making a solid composition for transmucosal delivery of Vitamin B12 material without the need of intrinsic factor comprising adding to the composition Vitamin B12 material and ingredients appropriate for the solid composition and making the solid composition, wherein the ingredients comprise, at least one bifunctional macromolecule with hydrophilic exterior and with hydrophobic pocket capable of pocketing Vitamin B12 material, at least one permeation/penetration enhancer and at least one agent that is mucoadhessive as well as permeation/penetration enhancer.

The process of making the sublingual film comprises following steps: (i) accurately weighing quantities of hydroxypropyl methyl cellulose or any other film forming ingredient/polymer, and excipients and dissolving them in water, (ii) mixing separately in water Methylcobalamin or other Vitamin B12 material, Isopropyl Myristate and [symbol] -Cyclodextrin and mixing this solution with solution prepared in above step i., (iii) coating the resulting solution on a support to the desired thickness into a film, (iv) allowing the film to dry at room temperature and cutting the same into suitable size so that each film contained selected quantity of methylcobalamin.

The process of making the tablet comprising following steps: (i) adding Methylcobalamin or other Vitamin B12 material to isopropyl alcohol or another carrier and mixing well by stirring, (ii) to the solution prepared in step i, further adding Magnesium Aluminium Silicate, [symbol] -cyclodextrin, isopropyl myristate, chitosan & sucralose or any other high intensity sweetener with stirring until the ingredients dissolve, (iii) adsorbing the resulting solution on the mixture of croscarmellose sodium or any other adsorbent, colloidal silicon dioxide, microcrystalline cellulose and mannitol. (iv) passing the powder blend was passed through a sieve #16 and then drying, (v) mixing the dried granules retained on the sieve #18, along with 15% fines with weighed amounts of lubricant and glidant and compressing to obtain orally disintegrating tablets. The process of making lozenges comprising following steps: (i) adding Methylcobalamin or any other Vitamin B12 material in propylene glycol or any other plasticizer and stirring for a period of time until a solution is obtained, (ii) to the solution prepared in step i, adding Magnesium Aluminium Silicate, [symbol] -cyclodextrin, isopropyl myristate, chitosan & sucralose or any other high intensity sweetener and stirring for a period of time until dissolution is achieved, (iii) adsorbing the mixture on mannitol, (iv) granulating the blend using solution of Hydroxypropyl Methyl C or any other binder, (v) passing the granules through sieve #16 and then drying, (vi) Mixing the dried granules retained on the sieve #18, along with 15% fines with weighed amounts of lubricant and glidant and compressing the tablets in machine to obtain tablet lozenges.

### DETAILED DESCRIPTION OF THE INVENTION

The instant invention has explored alternative and more efficient means for delivery of Vitamin B12 materials. The Vitamin B12 materials comprise cyanocobalamin, hydroxocobalamin and methyl cobalamin.

It is known that cyclodextrins are cyclic oligosaccharides with hydrophilic exterior and hydrophobic cavities. They can form soluble inclusion complexes with insoluble drugs. They have also been used as mucosal permeation/penetration enhancers for hydrophobic substances. Beta-Cyclodextrin has been used in the present case to form a 1:1 (cyclodextrin: Methylcobalamin) molar complex which is water soluble and better able to penetrate the buccal mucosa as compared to free drug.

It is also known that chitosan is a safe natural cationic polymer that has long been researched for its permeation enhancement properties and mucoadhesive properties. As permeation/penetration enhancer it opens tight junctions of cell membranes as shown by decrease of trans-epithelial electrical resistance. It interacts with the negatively charged mucus covering the mucus membranes and aids drug penetration by mucoahesion and thermodynamic activation of the mucosa.

Accordingly, it is also known that isopropyl myristate has been used many times as permeation/penetration enhancers in many of the pharmaceutical formulations.

In the present invention, it was found that sublingual films comprising methylcobalamine and further containing (a) Chitosan+ [symbol] -Cyclodextrin, or (b) Isopropyl myristate and [symbol] -cyclodextrin or (c) Chitosan and Isopropyl maleate did not facilitate sublingual permeation of methyl cobalamin.

Surprisingly, however, it is only when Chitosan, Isopropyl myristate and G-cyclodextrin are present together the sublingual absorption of methylcobalamin increased substantially. Usually the increase was almost about four times. It was observed that this combination of bifunctional agent, permeation/penetration enhancer/s and mucoadhesive agent/s can be used to make dosage forms for sub-lingual delivery. The dosage forms include, without limitation, a lozenge, a candy, a wafer, a tablet, a patch, a film, or a spray.

This invention has been illustrated in the form of sub-lingual oral film, a tablet and a lozenge. Process of making other dosage forms well known to a person skilled in the art can be used to make those dosage forms using the combination of at least one bifunctional macromolecule with hydrophilic exterior and with hydrophobic pockets capable of pocketing Vitamin B12 material, one permeation/penetration enhancer and one agent that is mucoadhessive as well as permeation/penetration enhancer as essential ingredients. In this invention, a thin polymeric film of small dimensions comprising water soluble fast dissolving polymer has been used as drug delivery system for delivering the above mentioned ‘bifunctional agent-permeation/penetration enhancer-mucoadhesive agent-drug-complex to the mucus membrane. It is an embodiment of this invention that the chitosan and Isopropyl myristate has been used in optimized concentration to aid drug penetration by enhancing the transport of cyclodextrin drug complex across the mucosa. The composition of this invention may comprise cyclodextrin in a range of 2.5% to 15% %, Isopropyl Myristate in a range of 0.5% to 15% and chitosan in a range of 1% to 15%. A person skilled in the art would understand that cyclodextrin can be replaced by a bifunctional agent of similar molecular nature that has hydrophilic exterior and hydrophobic pockets comprising unsubstituted or substituted derivatives of cyclodextrin including but not limited to -cyclodextrin, [symbol] -cyclodextrin, [symbol] cyclodextrin, Hydroxypropyl- [symbol] -cyclodextrin, methyl- [symbol] -cyclodextrin; the Isopropyl Myristate can be replaced by other penetration enhancer/s comprising fatty acid esters having permeation enhancement ability including but not limited to esters of oleic acid, linoleic acid, linolenic acid, hydroxyl fatty acids etc. and Chitosan can be replaced by other mucoadhesive permeation/penetration enhancer/s comprising substituted polysaccharides including but not limited to trimethyl chitosan (TMC), dimethylethyl chitosan (DMEC), diethylmethyl chitosan (DEMC), triethyl chitosan (TEC) and any derivative of chitosan.

Thus the present invention embodies one or more of following advantageous features—
1) Increasing the bioavailability of methylcobalamin by mechanisms independent of intrinsic factor
2) Capable of being delivered to any mucosal membrane as it is bioadhesive in nature
3) Can be consumed orally without the need for water.
4) Delivers complex in format with high surface area than other delivery forms which further helps to enhance dissolution and penetration of drug from delivery system by increased contact with mucosa and mucus.
5) Delivers the complex in a stable solid format. The complex is released in-vivo as the film dissolves. This feature overcomes the probability of destabilization of complex which is greater in liquid systems.
6) Delivers drug in a solid but thin and flexible format which is more patient friendly than dosage forms such as a tablet that is slowly dissolving in a sensitive mucosal area.
7) Can be cut into desired size and hence accuracy in delivery of dose can be achieved.
8) Unit dose can be packed using simple means like pouch or strip packing. No need of sophisticated packaging system as is required in the case of metered sprays etc.

DRI of Vitamin B₁₂ is provided in ‘Dietary Reference Intakes for Thiamin, Riboflavin, Niacin, Vitamin B6, Folate, Vitamin B12, Pantothenic Acid, Biotin-National Academic Press, available on the Internet link: http://www.nap.edu/catalog/6015.html.

This invention is illustrated by following non-limiting example/s. Variations and equivalents to the disclosed examples/embodiments shall be immediately apparent to a person skilled in the art and they are also considered to be included within the scope of this invention.

### EXAMPLE 1: PREPARATION OF METHYLCOBALAMIN SUBLINGUAL FILMS

| | Amount in percent | | | | | | | |
|---|---|---|---|---|---|---|---|---|
| Ingredients | 1 | 2 | 3 | 4 | 5 | 6 | 7 | 8 |
| Methylcobalamine | 5 | 4.68 | 4.75 | 4.68 | 4.47 | 4.41 | 4.47 | 4.23 |
| Propylene Glycol | 33.33 | 31.2 | 31.7 | 31.2 | 29.8 | 29.4 | 29.8 | 28.2 |
| Sucralose | 3.33 | 3.12 | 3.17 | 3.12 | 3.0 | 2.94 | 3.0 | 2.82 |
| Hydroxypropyl methyl cellulose | 51.61 | 46.87 | 49.2 | 48.36 | 46.19 | 45.6 | 46.19 | 43.7 |
| Magnesium aluminium silicate | 6.66 | 6.24 | 6.4 | 6.25 | 5.96 | 5.88 | 5.96 | 5.63 |
| IsoPropyl Myristate | — | 6.24 | — | — | 5.96 | 5.88 | — | 5.63 |
| Chitosan | — | — | 4.76 | — | 4.47 | — | 4.47 | 4.23 |
| [symbol] -Cyclodextrin | — | — | — | 6.25 | — | 5.88 | 5.96 | 5.63 |
| Weight of the each film | 30 mg | 32 mg | 31.5 mg | 32 mg | 33.5 mg | 34 mg | 33.5 mg | 35.5 mg |
| Drug Content | 1.5 mg | 1.5 mg | 1.5 mg | 1.5 mg | 1.5 mg | 1.5 mg | 1.5 mg | 1.5 mg |
| Disintegration time (In Seconds) | 18 | 29 | 15 | 17 | 17 | 22 | 24 | 21 |
| Dissolution Rate (Dissolution | 26.0 | 18.8 | 69.0 | 22.5 | 22.7 | 65.5 | 49.2 | 60.9 |
| medium: 500 ml of pH 6.8 | | | | | | | | |
| buffer, Time point 10 minutes) | | | | | | | | |

Procedure
1. Accurately weighed quantities of hydroxypropyl methyl cellulose, propylene glycol, magnesium aluminum silicate, and sucralose were mixed properly with the help of a stirrer in 50 ml of water.
2. Methylcobalamin along with remaining ingredients (As per the formula given in Table 1) was dissolved separately in 10 ml of water and mixed with solution prepared in step 1.
3. Resulting solution was casted on a support with the help of Stainless Steel blade to the desired thickness into films.
4. This film was then allowed to dry at room temperature and cut into suitable size so that each film contained 1.5 mg of methylcobalamin.
5. The films were characterized and the results are given in Table 1.

### EXAMPLE 2: ESTIMATION OF BLOOD LEVELS IN RATS

1. Studies were conducted on Sprague dawley rats. Animals were divided into three groups of four animals in each group.
2. Sublingual films of Formulation 3, 6 and 8 were selected for studying the in vivo release of methylcobalamin.
3. Animals were anesthetized and a strip was placed under the tongue of each animal. The strip being mucoadhesive adhered to the sublingual mucoas. Blood samples were collected at 5, 15, 30, 60, 120, 240, 360, 720 and 1440 min after dosing in heparinized eppendorf tubes. Samples were centrifuged at 10000 rpm for 10 min in cooling centrifuge (2-4° C.) and plasma was separated. Samples were then analyzed using optimized chromatographic method.
4. Formulation 3 produced Cmax of 0.125 ě 0.089 ιg/ml in 240 minutes, Formulation 6 produced Cmax of 0.197 ě 0.047 ιg/ml in 240 minutes and Formulation 8 produced C max of 0.410 ě 0.074 ιg/ml in 240 minutes.

### EXAMPLE 3: STUDY OF BLOOD LEVELS OF METHYLCOBALAMIN SUBLINGUAL FILMS (FORMULATION 8) IN HUMAN VOLUNTEERS

A single dose study in six human volunteers was carried out by giving one film to each volunteer by sublingual route. The volunteers were asked to hold the film under the tongue until it was completely dissolved. Blood samples were withdrawn at 0, 1, 2, 4, 8, 9, 10, 12, 24 hours. Blood samples were analyzed for the methylcobalamin content using AUTOPLEX ELISA & CLIA ANALYZER method. Methylcobalamin blood levels before and after dosing are given below:

| Volunteer | Blood levels before | Highest Blood levels | Percent |
|---|---|---|---|
| No. | dosing (pg/mL) | after dosing (pg/mL) | Increase |
| #1 | 437 | 585 | +133% |
| #2 | 197 | 593 | +301% |
| #3 | 178 | 648 | +364% |
| #4 | 182 | 504 | +276% |
| #5 | 183 | 349 | +190% |
| #6 | 138 | 414 | +300% |

### EXAMPLE 4: PREPARATION OF METHYLCOBALAMIN SUBLINGUAL TABLETS

| Ingredients | Amount in % |
|---|---|
| Methylcobalamin | 2.308 |
| Croscarmellose Sodium | 5.000 |
| Colloidal silicon dioxide | 0.520 |
| Microcrystalline cellulose | 29.390 |
| Mannitol | 32.820 |
| Propylene glycol | 15.385 |
| Veegum-F | 3.077 |
| [symbol] ⁻cyclodextrin | 3.077 |
| Isopropyl myristate | 3.077 |
| Chitosan | 2.308 |
| Sucralose | 1.538 |
| Talcum | 1.000 |
| Magnesium stearate | 0.500 |

Procedure:
1. Methyl cobalamin was added in propylene glycol and was stirred for 30 minutes.
2. To the solution prepared in step 1, Veegum-F, [symbol] -cyclodextrin, isopropyl myristate, chitosan & sucralose were added and stirring was continued for 30 minutes.
3. This solution was then adsorbed on the mixture of croscarmellose sodium, colloidal silicon dioxide, microcrystalline cellulose and mannitol.
4. The powder blend was passed through sieve #16 and then dried.
5. The dried granules retained on the sieve #18, along with 15% fines were mixed with weighed amounts of lubricant and glidant and were compressed in tabletting machine to obtain a compact flat faced orally disintegrating tablets.
6. The tablets were disintegrated in less than 30 seconds.

### EXAMPLE 5: FORMULATION AND DEVELOPMENT OF METHYLCOBALAMIN LOZENGES

| Ingredients | Amount in % |
|---|---|
| Methylcobalamin | 2.308 |
| HPMC (Methocel E5 Celsential) | 5.000 |
| Mannitol | 33.576 |
| Propylene glycol | 15.385 |
| Veegum-F | 3.077 |
| [symbol] ⁻cyclodextrin | 3.077 |
| Isopropyl myristate | 3.077 |
| Chitosan | 2.308 |
| Sucralose | 1.538 |
| Talcum | 1.000 |
| Magnesium stearate | 0.500 |
| Purified water* | Quantity sufficient |

Procedure:
1. Methylcobalamin was added in propylene glycol and was stirred for 30 minutes.
2. To the solution prepared in step 1, veegum-F, [symbol] -cyclodextrin, isopropyl myristate, chitosan & sucralose were added and stirred continuously for 30 minutes.
3. Above mixture was then adsorbed on mannitol. The blend was granulated using binder solution of HPMC.
4. The granules were passed through sieve #16 and then dried.
5. The dried granules retained on the sieve #18, along with 15% fines were mixed with weighed amounts of lubricant and glidant and were compressed in tabletting machine to obtain a compact flat faced tablet lozenges.

1. A solid composition of Vitamin B12 for transmucosal delivery without the need of intrinsic factor; the composition comprising Vitamin B12 and, at least one bifunctional macromolecule with hydrophilic exterior and with hydrophobic pockets capable of pocketing Vitamin B12 material, at least one permeation/penetration enhancer and at least one agent that is mucoadhessive as well as permeation/penetration enhancer.

2. The solid composition of Vitamin B12 of claim 1 wherein:
a. the Vitamin B12 material comprises, at least one or more, selected from the group consisting of cyanocobalamin, hydroxocobalamin and methylcobalamin.
b. the macromolecule with hydrophilic exterior and with hydrophobic pockets capable of pocketing Vitamin B12 material comprises, at least one or more, selected from the group consisting of cyclodextrin, -cyclodextrin, [symbol] -cyclodextrin, [symbol] cyclodextrin, Hydroxypropyl- [symbol] -cyclodextrin and methyl- [symbol] -cyclodextrin,
c. the permeation enhancer comprises, at least one or more, selected from the group consisting of Isopropyl Myristate, glycerol myristate, myristic acid and their derivatives or any other fatty acid esters that has permeation enhancement ability.
d. the mucoadhesive as well as penetration enhancer comprises, at least one or more, selected from the group consisting of chitosan, trimethyl chitosan (TMC), dimethylethyl chitosan (DMEC), diethylmethyl chitosan (DEMC), triethyl chitosan (TEC) and any derivative of chitosan or any substituted polysaccharides that have both mucoadhesive and permeation enhancement ability.

3. The solid composition of Vitamin B12 of claim 2 comprising a lozenge, a candy, a wafer, a tablet, a patch, a film, a spray, a lip balm, or gum.

4. The solid composition of Vitamin B12 of claim 3 wherein:
a. the film is a sub-lingual film further comprising a film forming polymer, propylene glycol or any other plasticizer, sucralose or any other high intensity sweetener, and Magnesium aluminium silicate or any other antisticking, anti-tacky agent,
b. the tablet or a lozenge further comprises a bulking agent, a disintegrant, a lubrincant, a high intensity sweetener, binder, anti adherent and other excipient/s.

5. The solid composition of Vitamin B12 comprising:
a. [symbol] -Cyclodextrin 2.5 to 15% of the composition,
b. Isopropyl Myristate 0.5 to 15-% of the composition,
c. chitosan 1- to 15% of the composition.

6. A method of transmucosal delivery of process of Vitamin B12 without the need of intrinsic factor comprising administering a solid composition comprising Vitamin B12 material comprising, at least one bifunctional macromolecule with hydrophilic exterior and with hydrophobic pockets capable of pocketing Vitamin B12 material, at least one permeation enhancer and at least one agent that is mucoadhessive as well as penetration enhancer.

7. The method of claim 6 wherein the solid composition of Vitamin B12 comprises:
a. the Vitamin B12 material further comprising at least one or more, selected from the group consisting of cyanocobalamin, hydroxocobalamin and methylcobalamin.
b. the macromolecule with hydrophilic exterior and with hydrophobic pockets capable of pocketing Vitamin B12 material comprises, at least one or more, selected from the group consisting of cyclodextrin-cyclodextrin, [symbol] -cyclodextrin, [symbol] cyclodextrin, Hydroxypropyl- [symbol] -cyclodextrin and methyl- [symbol] -cyclodextrin,
c. the permeation enhancer comprises, at least one or more, selected from the group consisting of Isopropyl Myristate, glycerol myristate, myristic acid and their derivatives or any other fatty acid esters that has permeation enhancement ability,
d. the mucoadhesive as well as penetration enhancer comprises, at least one selected from the group consisting of chitosan, trimethyl chitosan (TMC), dimethylethyl chitosan (DMEC), diethylmethyl chitosan (DEMC), triethyl chitosan (TEC) and any derivative of chitosan or any substituted polysaccharides that have both mucoadhesive and permeation enhancement ability.

8. The method of claim 7 wherein the solid composition of Vitamin B12 comprises a lozenge, a candy, a wafer, a tablet, a patch, a film, a spray, a lip balm, or gum.

9. The method of claim 8 wherein the solid composition of Vitamin B12 comprising:
a. the film is a sub-lingual film further comprising a film forming polymer, propylene glycol or any other plasticizer, sucralose or any other high intensity sweetener, and Magnesium aluminium silicate or any other antisticking, anti-tacky agent,
b. the tablet or a lozenge further comprises a bulking agent, a disintegrant, a lubrincant, a high intensity sweetener, binder, anti adherent and other excipient/s

10. The method of claim 9 wherein solid composition of Vitamin B12 comprises:
a. [symbol] -Cyclodextrin 2.5 to 15% of the composition,
b. Isopropyl Myristate 0.5 to 15-% of the composition,
c. chitosan 1- to 15% of the composition.

11. A process of making a solid composition for transmucosal delivery of Vitamin B12 material without the need of intrinsic factor comprising adding to the composition Vitamin B12 material and ingredients appropriate for the solid composition and making the solid composition, wherein the ingredients comprise, at least one bifunctional macromolecule with hydrophilic exterior and with hydrophobic pockets capable of pocketing Vitamin B12 material, at least one permeation enhancer and at least one agent that is mucoadhessive as well as penetration enhancer.

12. The process of claim 11 wherein the solid composition of Vitamin B12 comprises:
a. the Vitamin B12 material comprises at least one or more, selected from the group consisting of cyanocobalamin, hydroxocobalamin and methylcobalamin.
b. the macromolecule with hydrophilic exterior and with hydrophobic pockets capable of pocketing Vitamin B12 material comprises, at least one or more, selected from the group consisting of cyclodextrin-cyclodextrin, [symbol] -cyclodextrin, [symbol] cyclodextrin, Hydroxypropyl- [symbol] -cyclodextrin and methyl- [symbol] -cyclodextrin,
c. the permeation enhancer comprises, at least one or more, selected from the group consisting of Isopropyl Myristate, glycerol myristate, myristic acid and their derivatives or any other fatty acid esters that has permeation enhancement ability,
d. the mucoadhesive as well as penetration enhancer comprises, at least one selected from the group consisting of chitosan, trimethyl chitosan (TMC), dimethylethyl chitosan (DMEC), diethylmethyl chitosan (DEMC), triethyl chitosan (TEC) and any derivative of chitosan or any substituted polysaccharides that have both mucoadhesive and permeation enhancement ability.

13. The process of claim 12 wherein the solid composition of Vitamin B12 comprises a lozenge, a candy, a wafer, a tablet, a patch, a film, a spray, a lip balm, or gum.

14. The process of claim 13 wherein the solid composition of Vitamin B12 comprising:
a. the film is a sub-lingual film further comprising a film forming polymer, propylene glycol or any other plasticizer, sucralose or any other high intensity sweetener, and Magnesium aluminium silicate or any other antisticking, anti-tacky agent,
b. the tablet or a lozenge further comprises a bulking agent, a disintegrant, a lubrincant, a high intensity sweetener, binder, anti adherent and other excipient/s

15. The process of claim 14 comprising the sub-lingual film or the tablet or the lozenge comprising:
a. [symbol] -Cyclodextrin 2.5 to 15% of the composition,
b. Isopropyl Myristate 0.5 to 15-% of the composition,
c. chitosan 1- to 15% of the composition.

16. The process of claim 15 wherein:
a. the process of making the sublingual film comprises following steps:
i. accurately weighing quantities of hydroxypropyl methyl cellulose or any other film forming ingredient/polymer, and excipients and dissolving them in water,
ii. mixing separately in water Methylcobalamin or other Vitamin B12 material, Isopropyl Myristate and [symbol] -Cyclodextrin and mixing this solution with solution prepared in above step i.,
iii. coating the resulting solution on a support to the desired thickness into a film,
iv. allowing the film to dry at room temperature and cutting the same into suitable size so that each film contained selected quantity of methyl cobalamin.
b. the process of making the tablet comprising following steps:
i. adding Methylcobalamin or other Vitamin B12 material to isopropyl alcohol or another carrier and mixing well by stirring,
ii. to the solution prepared in step i, further adding Magnesium Aluminium Silicate, [symbol] -cyclodextrin, isopropyl myristate, chitosan & sucralose or any other high intensity sweetener with stirring until the ingredients dissolve,
iii. adsorbing the resulting solution on the mixture of croscarmellose sodium or any other adsorbent, colloidal silicon dioxide, microcrystalline cellulose and mannitol.
iv. passing the powder blend was passed through a sieve #16 and then drying,
v. mixing the dried granules retained on the sieve #18, along with 15% fines with weighed amounts of lubricant and glidant and compressing to obtain orally disintegrating tablets.
c. the process of making lozenges comprising following steps:
i. adding Methylcobalamin or any other Vitamin B12 material in propylene glycol or any other plasticizer and stirring for a period of time until a solution is obtained,
ii. to the solution prepared in step i, adding Magnesium Aluminium Silicate, [symbol] -cyclodextrin, isopropyl myristate, chitosan & sucralose or any other high intensity sweetener and stirring for a period of time until dissolution is achieved,
iii. adsorbing the mixture on mannitol,
iv. granulating the blend using solution of Hydroxypropyl Methyl C or any other binder,
v. passing the granules through sieve #16 and then drying,
vi. Mixing the dried granules retained on the sieve #18, along with 15% fines with weighed amounts of lubricant and glidant and compressing the tablets in machine to obtain tablet lozenges.
